(19) United States
(12) Patent Application Publication
(10) Pub. No.: US 2005/0288732 A1
(43) Pub. Date: Dec. 29, 2005

(54) Method and system to control skeletal muscles by means of neuro-electrical coded signals

(76) Inventors: Eleanor Schuler, Rio Rancho, NM (US); Claude Lee, Reno, NV (US); Dennis Meyer, Albuquerque, NM (US)

Correspondence Address: Ralph C. Francis;Francis Law Group, 1942 Embarcadero, Oakland, CA, 94606, US

(21) Appl. No.: 11/185,587

(22) Filed: Jul. 20, 2005

Related U.S. Application Data
(63) Continuation-in-part of application No. 10/871,928, filed on Jun. 18, 2004.
(60) Provisional application No. 60/592,751, filed on Jul. 30, 2004.
(60) Provisional application No. 60/602,438, filed on Aug. 18, 2004.
(60) Provisional application No. 60/604,279, filed on Aug. 24, 2004.
(60) Provisional application No. 60/479,407, filed on Jun. 18, 2003.

(51) Int. Cl. A61N 1/18

(52) U.S. Cl. 607/48; 607/68

(57) ABSTRACT

A method to control skeletal muscles generally comprising generating at least one coded waveform signal that is substantially similar to at least one coded waveform signal that is generated in the body and operative in the control of at least a first skeletal muscle and transmitting the generated waveform signal to a subject to control the first skeletal muscle.

### CROSS-REFERENCE TO RELATED APPLICATIONS

This application claims the benefit of U.S. Provisional Application Nos. 60/592,751, filed Jul. 30, 2004, 60/602,438, filed Aug. 18, 2004, and 60/604,279, filed Aug. 24, 2004 and is a continuation-in-part of U.S. application Ser. No. 10/871,928, filed Jun. 18, 2004, which claims the benefit of U.S. Provisional Application No. 60/479,407, filed Jun. 18, 2003.

### FIELD OF THE PRESENT INVENTION

The present invention relates generally to medical methods and systems for monitoring and controlling skeletal muscles. More particularly, the invention relates to a method and system for controlling skeletal muscles by means of transmitted neuro-electrical coded signals.

### BACKGROUND OF THE INVENTION

As is well known in the art, the brain modulates (or controls) skeletal muscles via electrical signals (i.e., action potentials or waveform signals), which are transmitted through the nervous system. The nervous system includes the central nervous system, which comprises the brain and the spinal cord, and the cranial and peripheral nervous systems, which generally comprise groups of nerve cells (i.e., neurons) and peripheral nerves that lie outside the brain and spinal cord. The various nerve networks and systems are anatomically separate, but functionally interconnected.

As indicated, the peripheral nervous system is constructed of nerve cells (or neurons) and glial cells (or glia), which support the neurons. Operative neuron units that carry signals from the brain are referred to as “efferent” nerves. “Afferent” nerves are those that carry sensor or status information to the brain. Together, these components of the nervous system are responsible for the function, regulation and modulation of the body's organs, muscles, secretory glands and other physiological systems.

As is known in the art, a typical neuron includes four morphologically defined regions: (i) cell body, (ii) dendrites, (iii) axon and (iv) presynaptic terminals. The cell body (soma) is the metabolic center of the cell. The cell body contains the nucleus, which stores the genes of the cell, and the rough and smooth endoplasmic reticulum, which synthesizes the proteins of the cell.

The nerve cell body typically includes two types of outgrowths (or processes); the dendrites and the axon. Most neurons have several dendrites; these branch out in tree-like fashion and serve as the main apparatus for receiving signals from other nerve cells.

The axon is the main conducting unit of the neuron. The axon carries coded electrical signals to the body's organs, skeletal muscles and other physiological systems to control the function thereof. The axon is capable of conveying electrical signals along distances that range from as short as 0.1 mm to as long as 2 m.

Near the end of the axon, the axon is divided into fine branches that make contact with other neurons. The point of contact is referred to as a synapse. The cell transmitting a signal is called the presynaptic cell. The cell receiving the signal is referred to as the postsynaptic cell. Specialized swellings on the axon's branches (i.e., presynaptic terminals) serve as the transmitting site in the presynaptic cell.

Most axons terminate near a postsynaptic neuron's dendrites. However, communication can also occur at the cell body or, less often, at the initial segment or terminal portion of the axon of the postsynaptic cell.

The electrical signals transmitted along the axon, referred to as action potentials, are rapid and transient “all-or-none” nerve impulses. Action potentials typically have an amplitude of less than approximately 100 millivolts (mV) and a duration of approximately 1 msec. Action potentials are conducted along the axon, without failure or distortion, at rates in the range of approximately 1-100 meters/sec. The amplitude of the action potential remains constant throughout the axon, since the impulse is continually regenerated as it traverses the axon.

As is known in the art, a “neurosignal” is a composite signal that includes many action potentials. The neurosignal also includes an instruction set for proper organ function and/or system. A skeletal muscle neurosignal would thus include an instruction set for a muscle to perform a desired movement, including information regarding initial muscle tension, degree of muscle movement, etc.

Neurosignals or “neuro-electrical coded signals” are thus codes that contain complete sets of information for complete organ function. As set forth in Co-Pending application Ser. No. 11/125,480, filed May 9, 2005, once these neurosignals, which are embodied in the “waveform signals” referred to herein, have been isolated, recorded, standardized and transmitted to a subject (or patient), a generated nerve-specific waveform instruction (i.e., waveform signal(s)) can be employed to control a skeletal muscle and, hence, treat a multitude of muscle impairments. The noted impairments include, but are not limited to, spinal injuries, brain tumor, multiple sclerosis, cerebral palsy, radiation-induced nerve damage, stroke induced neuron damage, etc.

As is known in the art, the contraction and movement of skeletal muscles is commanded and coordinated by a number of the aforementioned brain structures, including the cerebral cortex, cerebellum and brain system structures. To accomplish various brain designated tasks, neurosignals are transmitted to a target skeletal muscle or muscles to induce graduated coarse or fine motor movements.

Various apparatus, systems and methods have been developed, which include an apparatus for or step of recording action potentials or coded electrical neurosignals, to control various physiological systems. The signals are, however, typically subjected to extensive processing and are subsequently employed to operate and/or regulate a “mechanical” device or system, such as a muscle stimulator device. Illustrative are the systems disclosed in U.S. Pat. Nos. 6,360,740 and 6,651,652.

In U.S. Pat. No. 6,360,740, a system and method for providing respiratory assistance is disclosed. The noted method includes the step of recording “breathing signals”, which are generated in the respiratory center of a patient. The “breathing signals” are processed and employed to control a muscle stimulation apparatus or ventilator.

In U.S. Pat. No. 6,651,652, a system and method for treating sleep apnea is disclosed. The noted system includes a respiration sensor that is adapted to capture neuro-electrical signals and extract the signal components related to respiration. The signals are similarly processed and employed to control a ventilator.

In U.S. Pat. No. 5,167,229, a method and system for inducing skeletal muscle movement is disclosed. The method includes the step of implanting a sensor, i.e., input command means, in the body that is adapted to sense physical movement and provide a signal “which is indicative of a selected physiological movement or group of movements.” The signal is then processed and employed to control implanted electrodes that are adapted to stimulate target muscles.

A major drawback associated with the systems and methods disclosed in the noted patents, as well as most known systems, is that the control signals that are generated and transmitted are “user determined” and “device determinative”. The noted “control signals” are thus not related to or representative of the signals that are generated in the body and, hence, would not be operative in the control of the skeletal muscles if transmitted thereto.

It would thus be desirable to provide a method and system for controlling skeletal muscles that includes means for generating and transmitting coded electrical neurosignals (referred to herein as “waveform signals”) to the body that substantially correspond to the recorded waveform signals and are operative in the control of the skeletal muscles.

It is therefore an object of the present invention to provide a method and system for controlling skeletal muscles that overcomes the drawbacks associated with prior art methods and systems for controlling skeletal muscles.

It is another object of the invention to provide a method and system for controlling skeletal muscles that includes means for generating skeletal muscle waveform signals that substantially correspond to coded waveform signals that are generated in the body and are operative in the control of skeletal muscles.

It is another object of the invention to provide a method and system for controlling skeletal muscles that includes means for recording waveform signals that are generated in the body and operative in the control of skeletal muscles.

It is another object of the invention to provide a method and system for controlling skeletal muscles that includes processing means adapted to generate at least one base-line skeletal muscle signal that is representative of at least one coded waveform signal generated in the body from recorded waveform signals.

It is another object of the invention to provide a method and system for controlling skeletal muscles that includes processing means adapted to compare recorded skeletal muscle waveform signals to baseline skeletal muscle signals and generate a skeletal muscle signal based on the comparison of the signals.

It is another object of the invention to provide a method and system for controlling skeletal muscles that includes monitoring means for detecting skeletal muscle impairments and disorders.

It is another object of the invention to provide a method and system for controlling skeletal muscles that includes means for transmitting waveform signals to the body that substantially correspond to coded waveform signals that are generated in the body and are operative in the control of the skeletal muscles.

It is another object of the present invention to provide a method and system for controlling skeletal muscles that includes means for transmitting signals directly to the nervous system in the body that substantially correspond to coded waveform signals that are generated in the body and are operative in the control of the skeletal muscles.

It is another object of the invention to provide a method and system for controlling skeletal muscles that can be readily employed in the treatment of muscle and nerve related disorders and abnormalities, including spinal injuries and muscle nerve damage.

### SUMMARY OF THE INVENTION

In accordance with the above objects and those that will be mentioned and will become apparent below, the method to control skeletal muscles in one embodiment of the invention generally comprises (i) generating at least a first coded waveform signal that substantially corresponds to at least one coded waveform signal that is generated in the body and is recognizable by at least a first skeletal muscle as a control signal and (ii) transmitting the first waveform signal to a subject to control the first skeletal muscle.

In another embodiment of the invention, the method to control skeletal muscles generally comprises (i) capturing coded waveform signals that are generated in the body and are operative in the control of at least a first skeletal muscle, (ii) generating at least a first waveform signal that is recognizable by the first skeletal muscle as a control signal, and (iii) transmitting the first waveform signal to a subject to control the first skeletal muscle.

In one embodiment of the invention, the first waveform signal includes at least a second waveform signal that substantially corresponds to at least one of the captured waveform signals and is operative in the control of the first skeletal muscle.

In one embodiment of the invention, the first waveform signal is transmitted to the subject's nervous system. In another embodiment, the first waveform signal is transmitted proximate to a target zone on the neck, head or spinal region.

In another embodiment of the invention, the method to control skeletal muscles generally comprises (i) capturing coded waveform signals that are generated in the body and are operative in the control of skeletal muscles, (ii) storing the captured waveform signals in a storage medium, the storage medium being adapted to store the components of the captured waveform signals according to the function performed by the waveform signal components, (iii) generating at least a first waveform signal that substantially corresponds to at least one of the captured waveform signals and is operative in the control of at least a first skeletal muscle, and (iv) transmitting the first waveform signal to a subject to control the first skeletal muscle.

In another embodiment of the invention, the method to control skeletal muscles generally comprises (i) capturing a plurality of waveform signals generated in a first subject's body that are operative in the control of skeletal muscles, (ii) generating a base-line skeletal muscle waveform signal from the plurality of captured waveform signals, the base-line skeletal muscle waveform being operative in the control of a first skeletal muscle (iii) capturing a second waveform signal generated in the first subject's body that is operative in the control of the first skeletal muscle, (iv) comparing the base-line waveform signal to the second waveform signal, (v) generating a third waveform signal based on the comparison of the base-line and second waveform signals, and (vi) transmitting the third waveform signal to the first subject's body, the third waveform signal being operative in the control of the first skeletal muscle.

In one embodiment of the invention, the plurality of waveform signals is captured from a second subject's body.

In another embodiment of the invention, the plurality of waveform signals is captured from a plurality of subjects.

Preferably, the third waveform signal is transmitted to the subject's nervous system. In an alternative embodiment, the third waveform signal is transmitted proximate to a target zone on the neck, head or spinal region.

In accordance with a further embodiment of the invention, the method for controlling skeletal muscles generally comprises (i) monitoring the status of at least a first skeletal muscle of a subject, (ii) providing at least one skeletal muscle status signal in response to a skeletal muscle disorder of the first skeletal muscle, (iii) generating at least a first waveform signal that is operative in the control of the first skeletal muscle in response to the skeletal muscle status signal, and (iv) transmitting the first waveform signal to the subject to mitigate the skeletal muscle disorder.

In accordance with a further embodiment of the invention, the method for controlling skeletal muscles generally comprises (i) capturing waveform signals that are generated in the body and are operative in control of skeletal muscles, the waveform signals including at least a first waveform signal that is operative in the control of a first skeletal muscle, (ii) monitoring the skeletal muscle status of the first skeletal muscle of a subject and providing at least one skeletal muscle status signal indicative of the status of the first skeletal muscle, (iii) storing the captured waveform signals and skeletal muscle status signal in a storage medium, (iv) generating at least a first waveform that is operative in the control of the first skeletal muscle in response to a skeletal muscle status signal or component of a captured waveform signal that is indicative of a skeletal muscle disorder, and (v) transmitting the first waveform signal to the subject to mitigate the skeletal muscle disorder.

In yet another embodiment of the invention, the method to control skeletal muscles generally comprises (i) capturing a first plurality of waveform signals generated in the body that are operative in the control of skeletal muscles, (ii) capturing at least a first waveform signal from a subject's body that is indicative of a skeletal muscle disorder, (iii) generating a confounding signal that is operative to mitigate the skeletal muscle disorder, and (iv) transmitting the confounding waveform signal to the subject to mitigate the skeletal muscle disorder.

The system to control skeletal muscles, in accordance with one embodiment of the invention, generally comprises (i) at least a first signal probe adapted to capture coded waveform signals from the body, the waveform signals being representative of waveform signals naturally generated in the body and operative in the control of skeletal muscles, (ii) a processor in communication with the signal probe and adapted to receive the waveform signals, the processor being further adapted to generate at least a first waveform signal based on the captured waveform signals, the first waveform signal being recognizable by at least a first skeletal muscle as a control signal and (iii) at least a second signal probe adapted to be in communication with a subject's body for transmitting the first waveform signal to the body to control the first skeletal muscle.

Preferably, the processor includes a storage medium adapted to store the captured waveform signals.

In one embodiment, the processor is adapted to extract and store components of the captured waveform signals in the storage means according to the function performed by the signal components.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIGS. 1A through 1D are illustrations of waveform signals captured from the body that are operative in the control of the skeletal muscles of the arm forearm, hands and fingers;

FIG. 2 is an illustration of the skeletal muscles of the upper body (posterior view);

FIG. 3 is an illustration of the skeletal muscles of the right shoulder and chest regions (anterior view);

FIG. 4 is an illustration of the skeletal muscles of the right arm (anterior view);

FIG. 5 is a further illustration of the skeletal muscles of the right arm, showing the deep layer muscle structure (anterior view);

FIG. 6 is an illustration of the skeletal muscles of the right arm (posterior view);

FIG. 7 is a further illustration of the skeletal muscles of the right arm, showing the deep layer muscle structure (posterior view);

FIGS. 8A and 8B are illustrations of the skeletal muscles of the right forearm (posterior views);

FIG. 9 is an illustration of the skeletal muscles of the right hand (anterior view);

FIG. 10 is a schematic illustration of one embodiment of a skeletal muscle control system, according to the invention;

FIG. 11 is a schematic illustration of another embodiment of a skeletal muscle control system, according to the invention;

FIG. 12 is a schematic illustration of another embodiment of a skeletal muscle control system, according to the invention; and

FIG. 13 is a schematic illustration of yet another embodiment of a skeletal muscle control system, according to the invention.

### DETAILED DESCRIPTION OF THE INVENTION

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred systems and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a waveform signal” includes two or more such signals; reference to “a skeletal muscle disorder” includes two or more such disorders and the like.

Definitions

The term “nervous system”, as used herein, means and includes the central nervous system, including the spinal cord, medulla, pons, cerebellum, midbrain, diencephalon and cerebral hemispheres, and the cranial and peripheral nervous systems, including the neurons and glia.

The terms “coded waveform signal” and “waveform signal”, as used herein, mean and include a composite electrical signal that is generated in the body and carried by neurons in the body, including neurocodes, neurosignals and components and segments thereof.

The term “skeletal muscle”, as used herein, means and includes a striated muscle, normally having at least one attachment to the skeletal system, whose contraction and extension are controlled or mediated by cognitive action.

The term “target zone”, as used herein, means and includes, without limitation, a region of the body proximal to a portion of the nervous system whereon the application of electrical signals can induce the desired neural control without the direct application (or conduction) of the signals to a target nerve.

The terms “patient” and “subject”, as used herein, mean and include humans and animals.

The term “plexus”, as used herein, means and includes a branching or tangle of nerve fibers outside the central nervous system.

The term “ganglion”, as used herein, means and includes a group or groups of nerve cell bodies located outside the central nervous system.

The terms “skeletal muscle impairment” and “skeletal muscle disorder”, as used herein, mean and include any dysfunction of a skeletal muscle that impedes the normal function thereof. Such dysfunction can be caused by a multitude of known factors and events, including, without limitation, spinal cord injury and severance, a brain tumor, multiple sclerosis, cerebral palsy and involuntary muscle contractions.

The present invention substantially reduces or eliminates the disadvantages and drawbacks associated with prior art methods and systems for controlling skeletal muscles. In one embodiment of the invention, the system for controlling skeletal muscles generally comprises means for generating at least one waveform signal that substantially corresponds to at least one waveform signal (i.e., coded electrical neurosignal) that is generated in the body and is operative in the control of at least a first skeletal muscle and means for transmitting the waveform signal to a subject's body. In a preferred embodiment of the invention, the waveform signal is transmitted to the subject's nervous system.

In a further embodiment, the system includes means for recording waveform signals from a subject's body that are operative in the control of at least the first skeletal muscle. According to the invention, the “subject” can be the same subject that the generated waveform signals are transmitted to or a different subject.

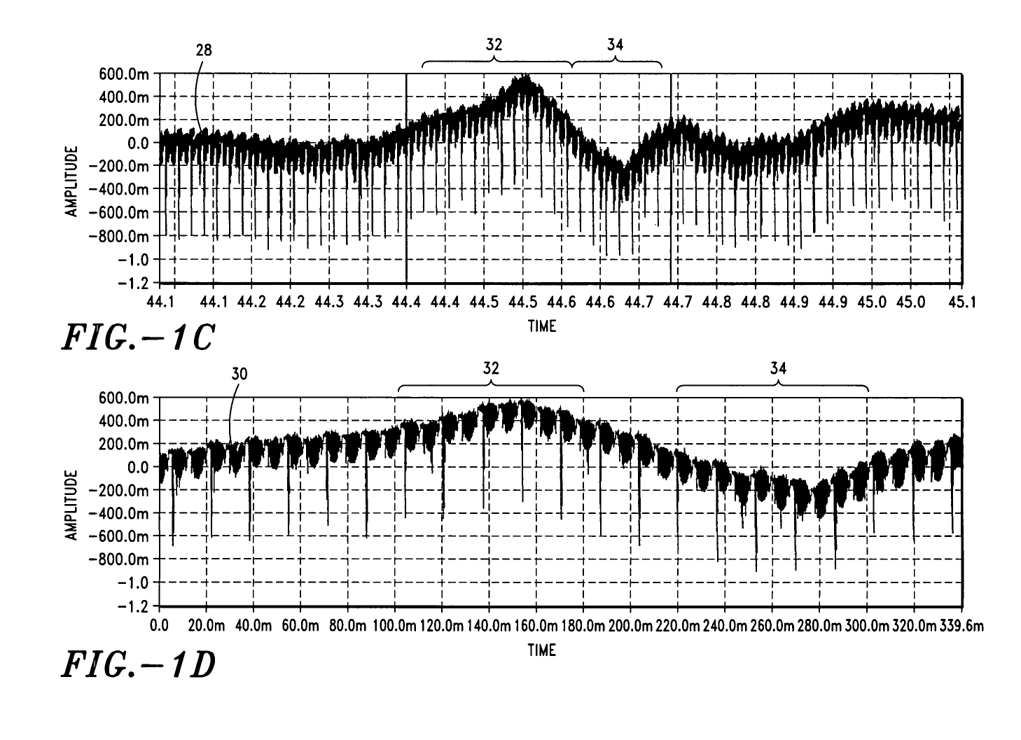

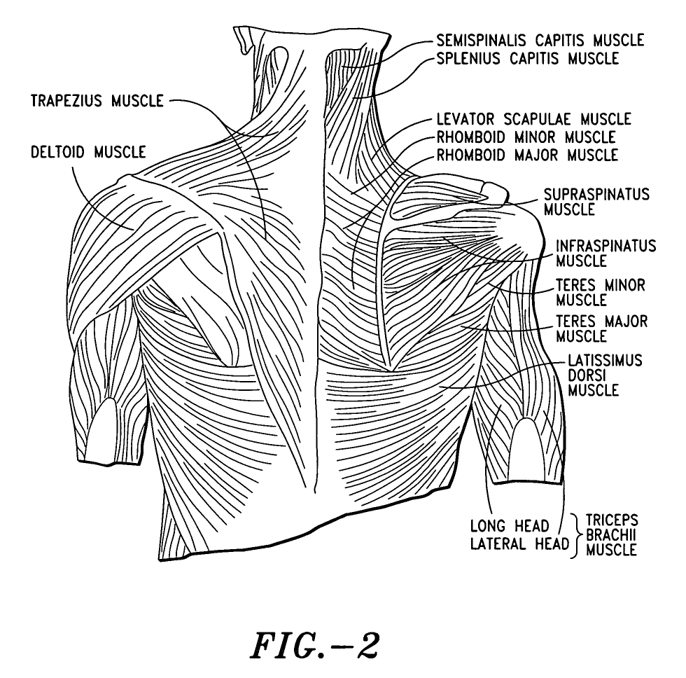

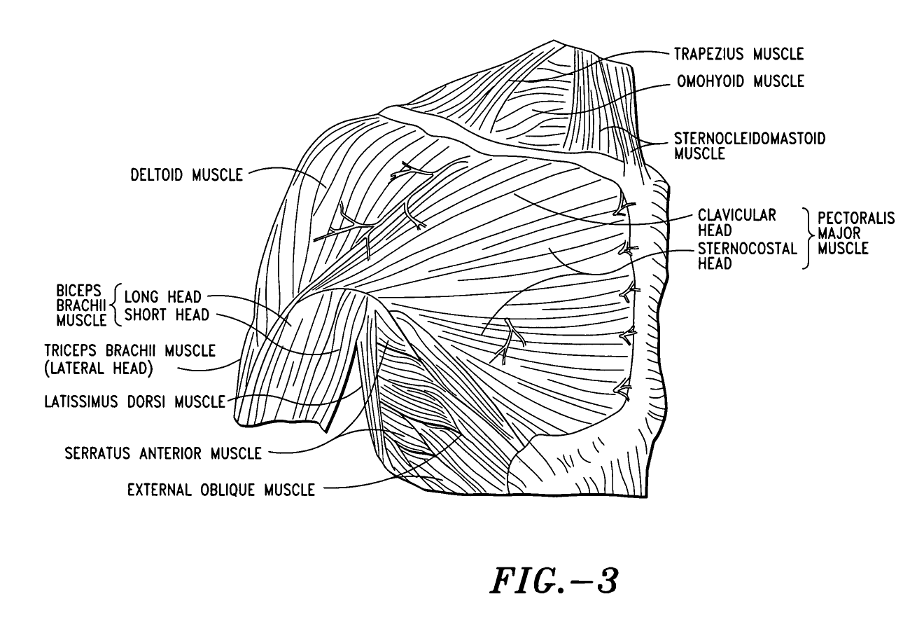

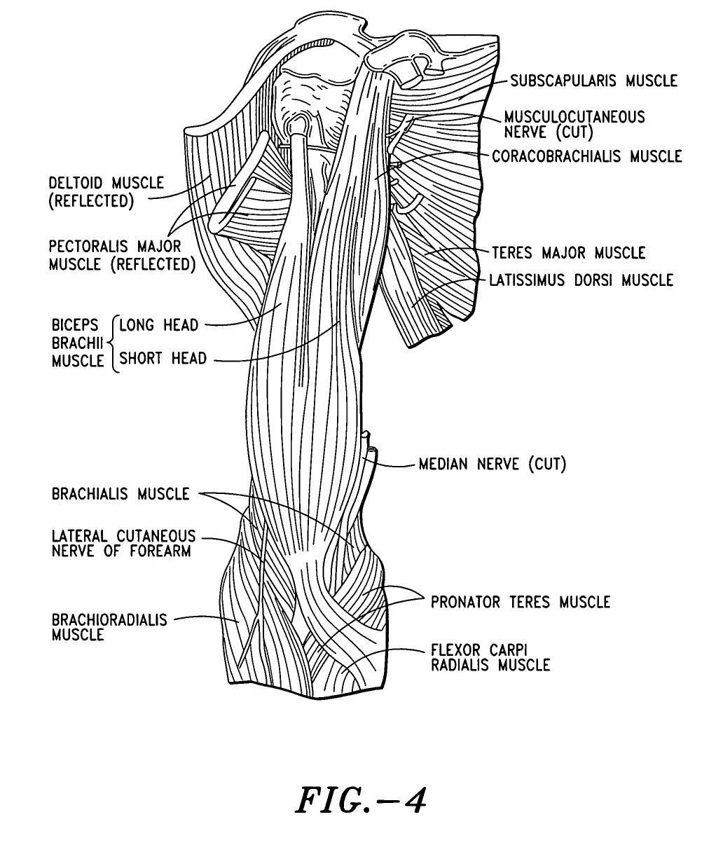

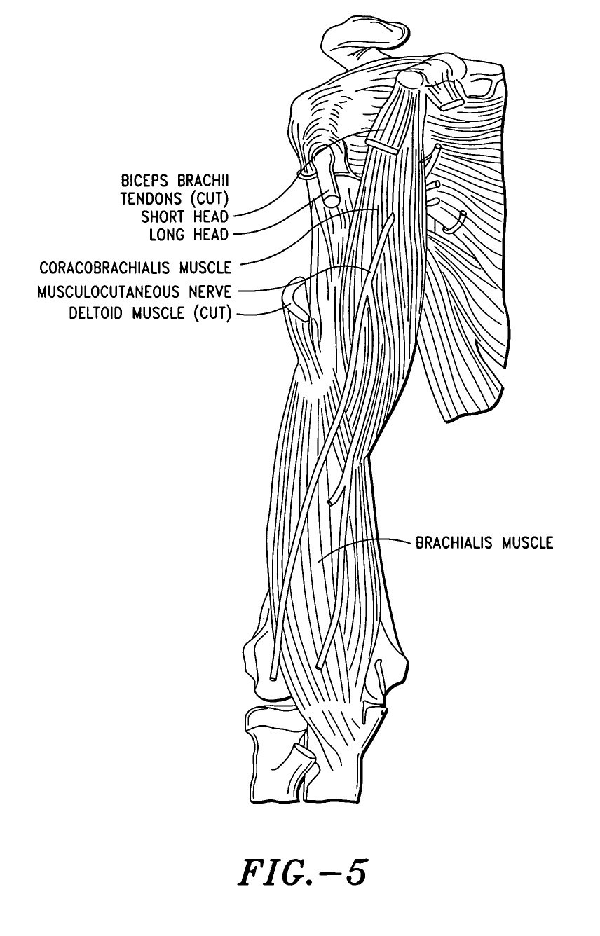

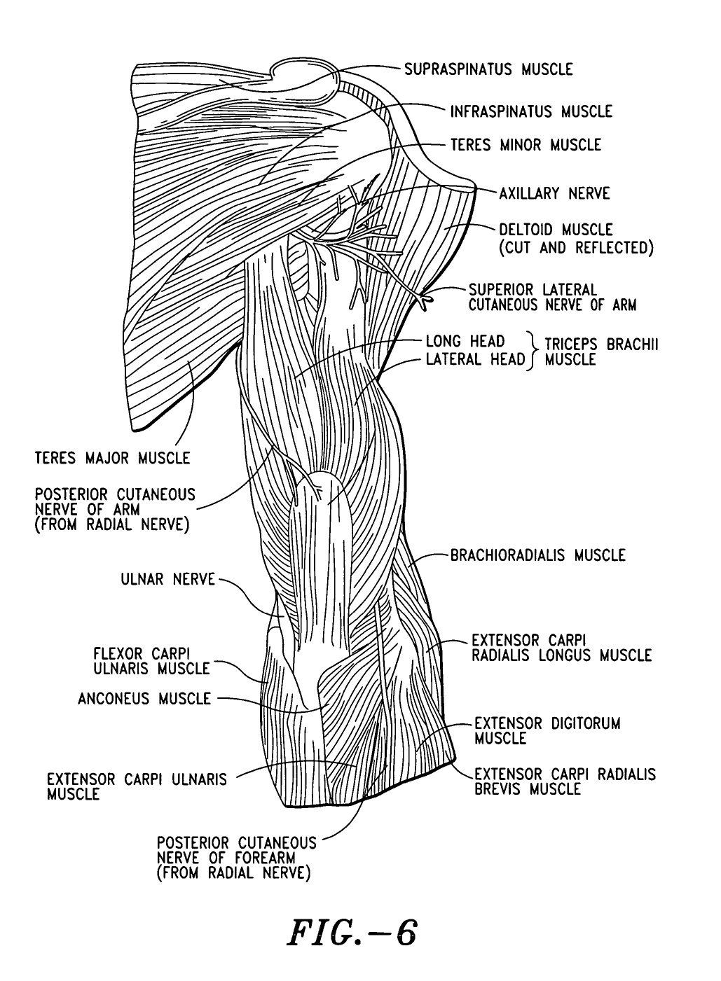

Referring now to FIGS. 2 through 9, there are shown illustrations of various skeletal muscles and muscle structures of the upper body, which can be controlled through the use of the methods and system of the invention. As illustrated in FIGS. 2 through 7, the skeletal muscles of the shoulder and upper arm include the levator scapulae, major and minor rhomboids, deltoids, supraspinatus, trapezius, pectoralis, coracobrachialis, biceps and triceps brachii, and latissimus dorsi.

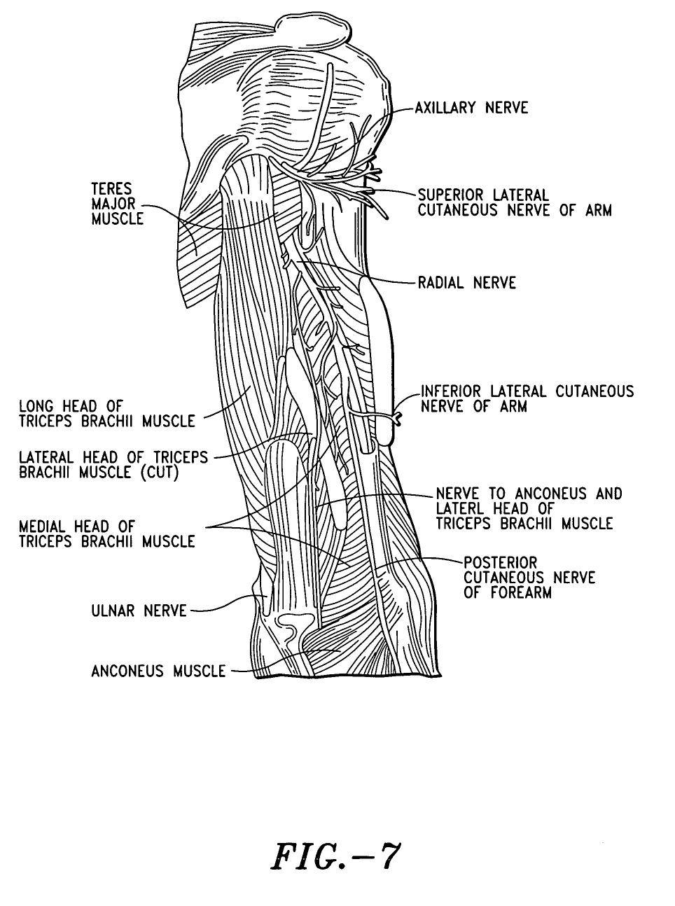

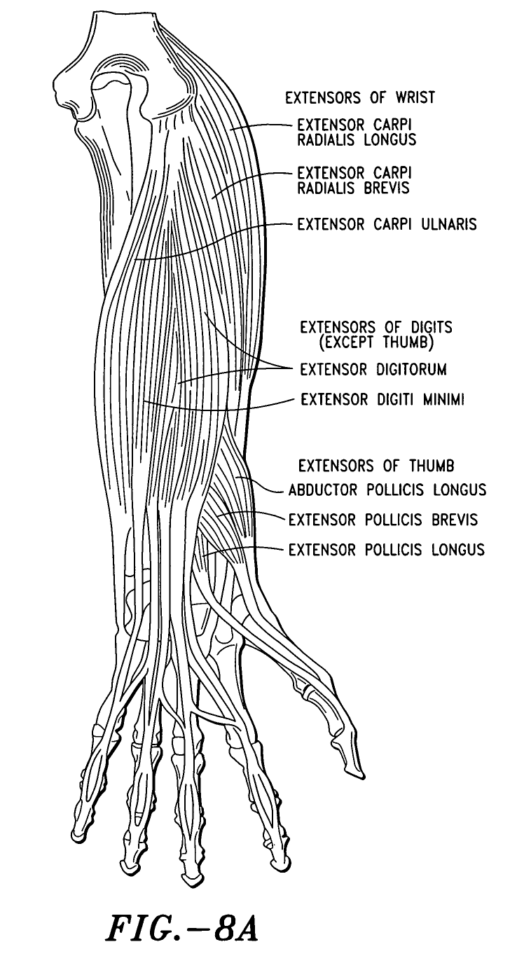

As illustrated in FIGS. 8A, 8B and 9, the skeletal muscles of the forearm, wrist and hand include the extensor and flexor digitorums, extensor carpi ulnaris, abductor and flexor pollicis longus, lumbrical, opponens and adductor pollicis muscles, and finger and wrist flexors.

It is to be understood that, although only the skeletal muscles of the upper body are illustrated, the skeletal muscles of the lower body are similarly within the scope of the present invention. Such skeletal muscles include, without limitation, the quadriceps, hamstrings, adductor longus, vastus lateralis, intermedius and medialis muscles, and the sartorius.

As indicated, coded waveform signals related to skeletal muscle operation and control originate in various brain structures. The waveform signals are primarily transmitted through the spinal cord. The waveform signals that control the noted skeletal muscles of the shoulder, arm, wrist and hand are also transmitted through the brachial plexus, and the radial, median and ulnar nerves.

According to the invention, the waveform signals that control a target skeletal muscle or muscles can be captured or collected along any of the nerves carrying the waveform signals to the target skeletal muscle. By way of example, the waveform signals transmitted to the abductor pollicis muscle of the hand can be captured from the brachial plexus.

Methods and systems for capturing coded signals from specific nerves in the body, and for storing, processing and transmitting neuro-electrical signals (or coded waveform signals) are set forth in Co-Pending application Ser. No. 10/000,005, filed Nov. 20, 2001, and application Ser. No. 11/125,480, filed May 9, 2005; which are incorporated by reference herein in their entirety.

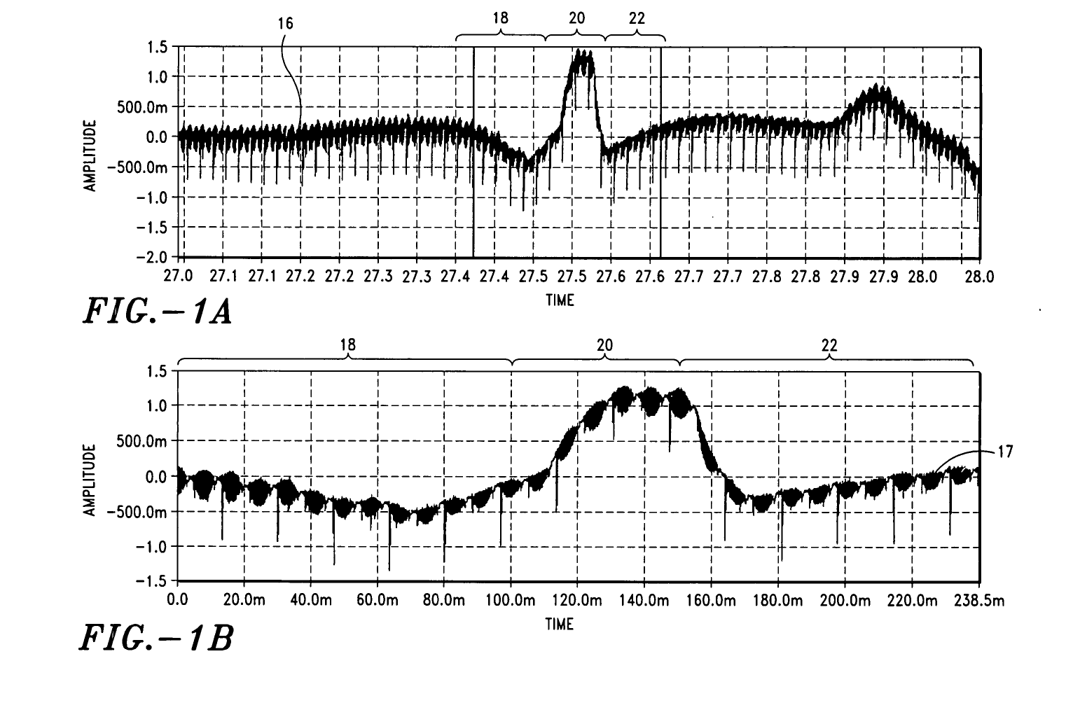

Referring now to FIGS. 1A through 1D, there are shown exemplar waveform signals that are operative in the control of the skeletal muscles of the arm, forearm, hands and fingers. The signals 16, 17 shown in FIGS. 1A and 1B bring the arm upward and pull the hand back with the fingers spread. The signals 28, 30 shown FIGS. 1C and 1D provide the same movement as the signals shown in FIGS. 1A and 1B with less intensity (i.e., moderate movement).

As illustrated in FIGS. 1A and 1B, each signal 16, 17 includes a negative segment 18, which is believed to reflect the muscle and/or nerve setting up for movement. Following the negative segment 18 is a large positive segment 20, which produces the desired movement, and a negative segment 22, thereafter reflecting the rest and evaluation segment of the signal.

As stated above, the noted signals include coded information related to muscle movement function, such as initial muscle tension, degree (or depth) of muscle movement, etc.

In accordance with one embodiment of the invention, coded waveform signals generated in the body that are operative in the control of skeletal muscles, such as the signals shown in FIGS. 1A and 1B, are captured and transmitted to a processor or control module. Preferably, the control module includes storage means adapted to store the captured signals. In a preferred embodiment, the control module is further adapted to store the components of the captured signals (that are extracted by the processor) in the storage means according to the function performed by the signal components.

According to the invention, the stored signals can subsequently be employed to establish at least one, preferably, multiple base-line skeletal muscle waveform signals. The module can then be programmed to compare skeletal muscle waveform signals (and components thereof) captured from a subject and, as discussed below, generate at least one waveform signal or modified base-line waveform signal for transmission to the same or a different subject. Such modification can include, for example, increasing the amplitude of a skeletal muscle signal to provide a quicker or more powerful muscle movement.

According to the invention, the captured waveform signals are preferably processed by proprietary means and a waveform signal (i.e., coded electrical neurosignal) that is representative of at least one captured waveform signal and operative in the control of at least one skeletal muscle (i.e., recognized by the brain or at least one skeletal muscle as a control signal) is generated by the control module. The noted waveform signal is preferably similarly stored in the storage means of the control module.

Methods and systems for processing coded waveform signals are set forth in co-pending application Ser. No. ______ [Attorney Docket No. SCM-02-019U], filed Jun. 10, 2005; which is incorporated by reference herein in its entirety.

In accordance with one embodiment of the invention, the generated waveform signal is accessed from the storage means and transmitted to the subject via a transmitter (or treatment member) to control a target skeletal muscle or muscles. As discussed in detail herein, various transmitters can be employed within the scope of the invention to transmit the generated waveform signals to a subject.

According to the invention, the applied voltage of a transmitted waveform signal (or signals) can be up to 20 volts AC (up to 3 volts DC) to allow for voltage loss during the transmission of the signals. Preferably, current is maintained to less than 2 amp output.

Direct conduction into the nerves via electrodes connected directly to such nerves preferably have outputs less than 3 volts AC and current less than one tenth of an amp.

As is known in the art and discussed in detail in Co-Pending application Ser. Nos. 11/125,480 and ______ [Attorney Docket No. SCM-02-019U], filed Jun. 10, 2005, varying the voltage of transmitted waveform signals causes movement changes, which are generally proportional to the voltage change. For example, a waveform signal delivered at a slightly higher voltage will cause a stronger and larger muscle movement. Likewise, the same waveform signal delivered at a slightly lower voltage will cause a lesser and smaller movement of the target muscle(s).

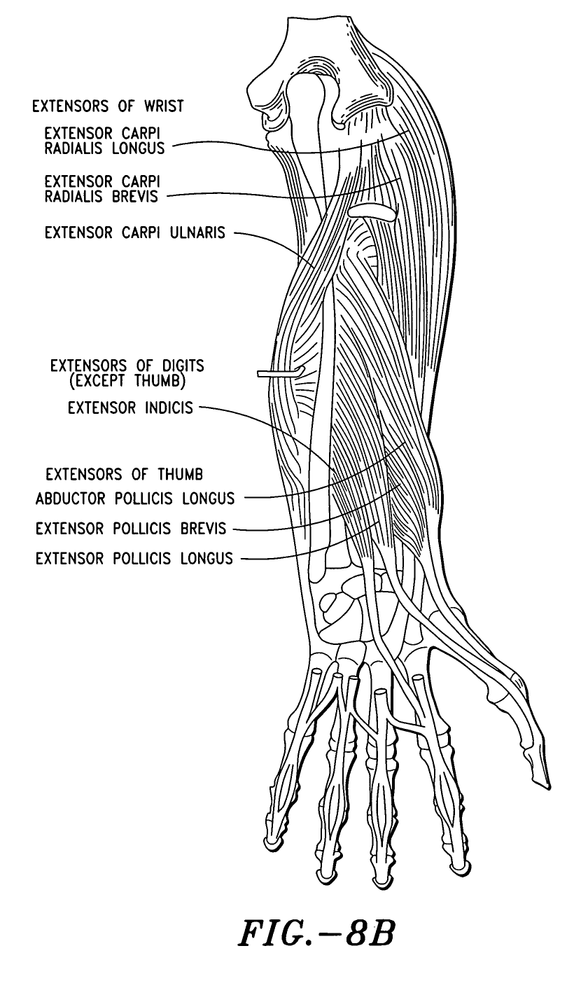

Referring now to FIG. 10, there is shown a schematic illustration of one embodiment of a skeletal muscle control system 20A of the invention. As illustrated in FIG. 10, the control system 20A includes a control module 22, which is adapted to receive coded neurosignals or “waveform signals” from a skeletal muscle signal sensor (shown in phantom and designated 21) that is in communication with a subject, and at least one treatment member 24.

The treatment member 24 is adapted to communicate with the body and receives generated waveform signals from the control module 22. According to the invention, the treatment member 24 can comprise an electrode, antenna, a seismic transducer, or any other suitable form of conduction attachment for transmitting skeletal muscle waveform signals that control skeletal muscle function in human and animals.

The treatment member 24 can be attached to appropriate nerves via a surgical process. Such surgery can, for example, be accomplished with “key-hole” entrance in a thoracic-stereo-scope procedure. If necessary, a more expansive thoracotomy or other surgical approach can be employed for placement of the treatment member 24.

As illustrated in FIG. 10, the control module 22 and treatment member 24 can be entirely separate elements, which allow system 20A to be operated remotely. According to the invention, the control module 22 can be unique, i.e., tailored to a specific operation and/or subject, or can comprise a conventional device.

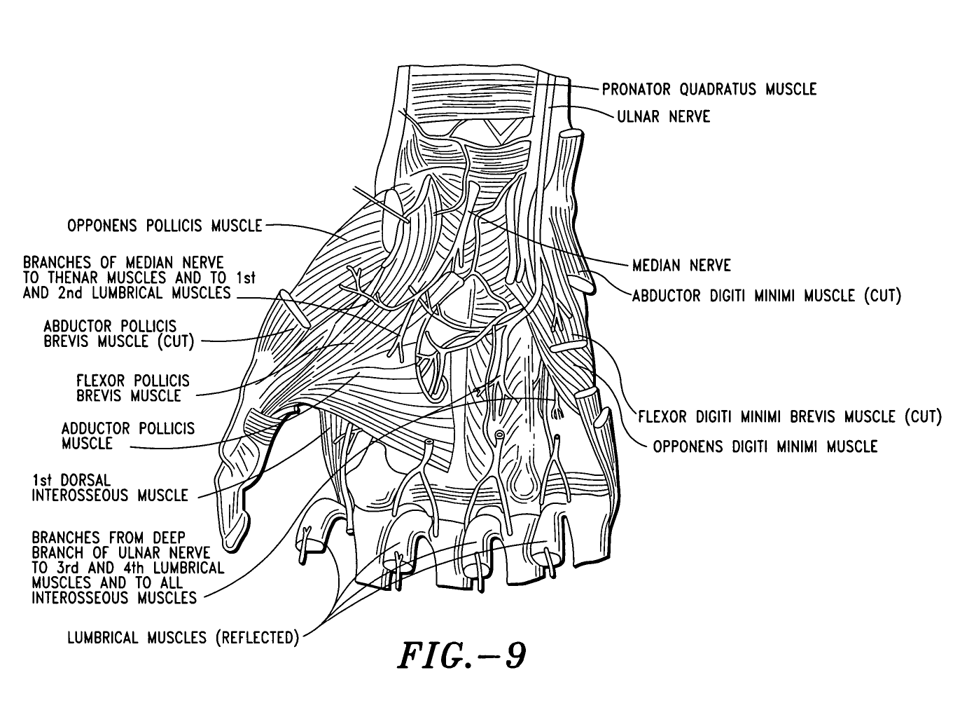

Referring now to FIG. 11, there is shown a further embodiment of a control system 20B of the invention. As illustrated in FIG. 11, the system 20B is similar to system 20A shown in FIG. 10. However, in this embodiment, the control module 22 and treatment member 24 are connected.

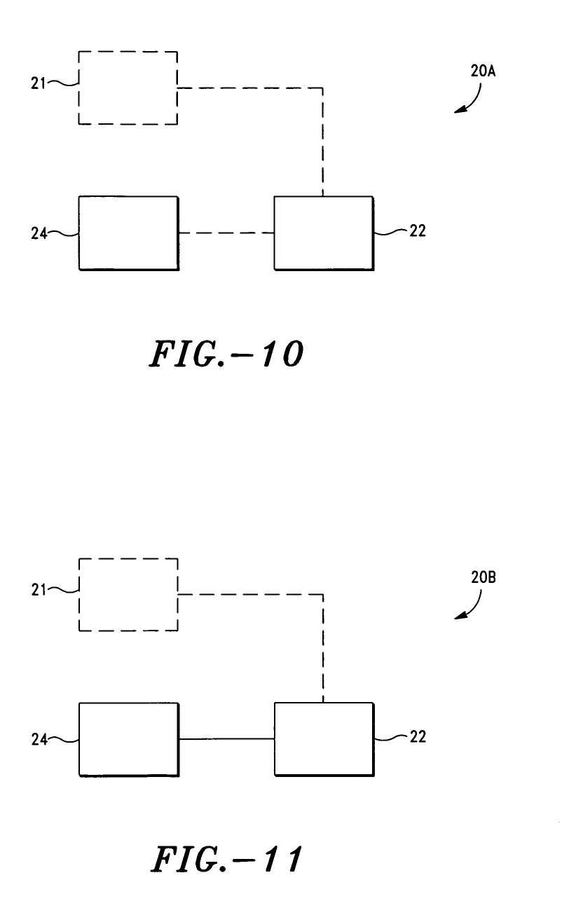

Referring now to FIG. 12, there is shown yet another embodiment of a control system 20C of the invention. As illustrated in FIG. 12, the control system 20C similarly includes a control module 22 and a treatment member 24. The system 20C further includes at least one skeletal muscle signal sensor 21.

The system 20C also includes a processing module (or computer) 26. According to the invention, the processing module 26 can be a separate component or can be a sub-system of a control module 22′, as shown in phantom.

As indicated above, the processing module (or control module) 26 preferably includes storage means adapted to store the captured skeletal muscle waveform signals. In a preferred embodiment, the processing module 26 is further adapted to extract and store the components of the captured skeletal muscle waveform signals in the storage means according to the function performed by the signal components.

In one embodiment of the invention, the method for controlling skeletal muscles includes the following steps: (i) generating at least a first coded waveform signal that substantially corresponds to at least one coded waveform signal that is generated in the body and is recognizable by at least a first skeletal muscle as a control signal and (ii) transmitting the first waveform signal to a subject to control the first skeletal muscle.

In one embodiment of the invention, the first waveform signal is transmitted to the subject's nervous system. In another embodiment, the first waveform signal is transmitted proximate to a target zone on the neck, head or spinal region.

According to the invention, the generated waveform signal is preferably transmitted to the subject via a constant current or constant voltage method.

The constant current method allows for the voltage level to fluctuate as the resistance changes. In one embodiment, a positive and negative probe (the negative probe located cranial to the positive probe) are attached to a target nerve. The distance between the probes is preferably approximately 2 cm. A ground connection is also made between the interior muscles and an earth ground.

In the constant voltage method, a signal probe is attached to the target nerve. While the signal probe is capable of providing both the positive and negative portions of the neuro-code, only the positive portion of the neuro-code is used to stimulate the nerve. The signal ground probe is not required. A ground connection is similarly made between the interior muscles and an earth ground.

In another embodiment of the invention, the method to control skeletal muscles generally comprises (i) capturing waveform signals that are generated in the body and are operative in the control of at least a first skeletal muscle, (ii) generating at least a first waveform signal that is recognizable by the first skeletal muscle as a control signal, and (iii) transmitting the first waveform signal to a subject to control the first skeletal muscle.

In a preferred embodiment, the first waveform signal includes at least a second waveform signal that substantially corresponds to at least one of the captured waveform signals and is operative in the control of the first skeletal muscle.

In one embodiment of the invention, the first waveform signal is transmitted to the subject's nervous system. In another embodiment, the first waveform signal is transmitted proximate to a target zone on the neck, head or spinal region.

In another embodiment of the invention, the method to control skeletal muscles generally comprises (i) capturing waveform signals that are generated in the body and are operative in control of skeletal muscles, (ii) storing the captured waveform signals in a storage medium, the storage medium being adapted to store the components of the captured waveform signals according to the function performed by the signal components, (iii) generating at least a first waveform signal that substantially corresponds to at least one of the captured waveform signals and is operative in the control of at least a first skeletal muscle, and (iv) transmitting the first waveform signal to a subject.

In another embodiment of the invention, the method to control skeletal muscles generally comprises (i) capturing a first plurality of waveform signals generated in a first subject's body that are operative in the control of skeletal muscles, (ii) generating a base-line skeletal muscle waveform signal from the first plurality of waveform signals, the base-line skeletal muscle waveform being operative in the control of a first skeletal muscle (iii) capturing a second waveform signal generated in the first subject's body that is operative in the control of the first skeletal muscle, (iv) comparing the base-line waveform signal to the second waveform signal, (v) generating a third waveform signal based on the comparison of the base-line and second waveform signals, and (vi) transmitting the third waveform signal to the first subject, the third waveform signal being operative in the control of the first skeletal muscle.

In one embodiment of the invention, the first plurality of waveform signals is captured from a second subject's body.

In another embodiment of the invention, the first plurality of waveform signals is captured from a plurality of subjects.

In one embodiment of the invention, the first and third waveform signals are transmitted to the subject's nervous system. In another embodiment, the first waveform signal is transmitted proximate to a target zone on the neck, head or spinal region.

According to the invention, the step of transmitting the waveform signals of the invention to a subject can be accomplished by direct conduction via attachment of an electrode to the receiving nerve or nerve plexus. As discussed, this requires a surgical intervention to physically attach the electrode to the selected target nerve.

In alternative embodiments of the invention, the step of transmitting the waveform signals of the invention to a subject can also be accomplished by transposing the waveform signal into a seismic form. The seismic signal is then sent into a region of the head, neck, or spinal region in a manner that allows the appropriate “nerve” to receive and obey the coded instructions of the seismic signal.

The present invention thus provides methods and apparatus to effectively control skeletal muscles. The methods and apparatus can thus be employed to restore some or most appendage (i.e., arm, hand and leg) movement in paralyzed subjects. The methods and apparatus of the invention can also be employed in the treatment of various skeletal muscle impairments or disorders, such as involuntary muscle contractions resulting from hypertonia and spasticity.

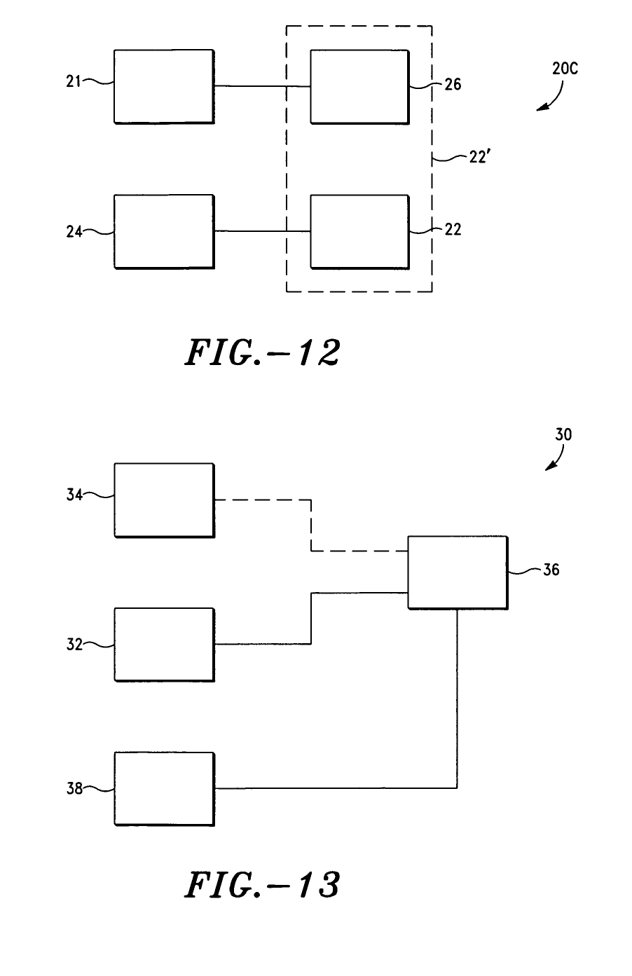

Referring now to FIG. 13, there is shown one embodiment of a skeletal muscle control system 30 that can be employed in the treatment of various skeletal muscle impairments and disorders. As illustrated in FIG. 13, the system 30 includes at least one skeletal muscle sensor 32 that is adapted to monitor the skeletal muscle function or status of at least a first skeletal muscle of a subject and transmit at least one signal indicative of the first skeletal muscle status.

According to the invention, the first skeletal muscle status can be determined by a multitude of factors, including skeletal muscle movement or lack thereof, muscle tension, etc. Various sensors can thus be employed within the scope of the invention to detect the noted factors and, hence, a skeletal muscle impairment or disorder.

The system 30 further includes a processor 36, which is adapted to receive the skeletal muscle status signal(s) from the skeletal muscle sensor 32. The processor 36 is further adapted to receive skeletal muscle waveform signals recorded by a skeletal muscle signal probe (shown in phantom and designated 34).

In a preferred embodiment of the invention, the processor 36 includes storage means for storing the captured, waveform signals and skeletal muscle status signals. The processor 36 is further adapted to extract the components of the waveform signals and store the signal components in the storage means.

In a preferred embodiment, the processor 36 is programmed to detect skeletal muscle status signals indicative of skeletal muscle impairments and/or disorders and/or waveform signals and components thereof indicative of skeletal muscle disorders and generate at least one waveform signal that is operative in the control of at least one skeletal muscle. Thus, in a preferred aspect of the noted embodiment, the processor 36 is programmed to detect a skeletal muscle status signal indicative of a first skeletal muscle disorder and generate at least a first waveform signal that is operative in the control of the first skeletal muscle, which, when transmitted to the subject (as discussed below) mitigates the first skeletal muscle disorder.

Referring to FIG. 13, the generated waveform signal is routed to a transmitter 38 that is adapted to be in communication with the subject's body. The transmitter 38 is further adapted to transmit the waveform signal to the subject's body (in a similar manner as described above) to control the affected skeletal muscle and, preferably, mitigate the detected skeletal muscle disorder.

According to the invention, the waveform signal is preferably transmitted to one or more nerves that are in communication with the affected skeletal muscle. A single waveform signal or a plurality of signals can be transmitted in conjunction with one another.

Thus, in accordance with a further embodiment of the invention, the method for controlling skeletal muscles generally comprises (i) monitoring the status of at least a first skeletal muscle of a subject, (ii) providing at least one skeletal muscle status signal in response to a skeletal muscle disorder of the first skeletal muscle, (iii) generating at least a first waveform signal that is operative in the control of the first skeletal muscle in response to the skeletal muscle status signal, and (iv) transmitting the first waveform signal to the subject to mitigate the skeletal muscle disorder.

In accordance with a further embodiment of the invention, the method for controlling skeletal muscles generally comprises (i) capturing waveform signals that are generated in the body and are operative in control of skeletal muscles, the waveform signals including at least a first waveform signal that is operative in the control of a first skeletal muscle, (ii) monitoring the skeletal muscle status of the first skeletal muscle of a subject and providing at least one skeletal muscle status signal indicative of the status of the first skeletal muscle, (iii) storing the captured waveform signals and skeletal muscle status signal in a storage medium, (iv) generating at least a first waveform that is operative in the control of the first skeletal muscle in response to a skeletal muscle status signal or component of a captured waveform signal that is indicative of a skeletal muscle disorder, and (v) transmitting the first waveform signal to the subject to mitigate the skeletal muscle disorder.

In yet another embodiment of the invention, the method to control skeletal muscles generally comprises (i) capturing a first plurality of waveform signals generated in the body that are operative in the control of skeletal muscles, (ii) capturing at least a first waveform signal from a subject's body that is indicative of a skeletal muscle disorder, (iii) generating a confounding signal that is operative to mitigate the skeletal muscle disorder, and (iv) transmitting the confounding waveform signal to the subject to mitigate the skeletal muscle disorder.

As will be appreciated by one having skill in the art, the present invention provides numerous advantages. Among the advantages are the provision of a system, apparatus and method to control skeletal muscles that can be readily and effectively employed in the treatment of various skeletal muscle impairments and disorders, including involuntary muscle movement (e.g., spasms and muscle contractions) and partial or full loss of muscle movement or control resulting from spinal injuries, multiple sclerosis, cerebral palsy, radiation-induced nerve damage, stroke induced neuron damage, etc.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A method for controlling skeletal muscles, comprising the steps of:
generating at least a first waveform signal that is recognizable by at least a first skeletal muscle as a control signal; and
transmitting said first waveform signal to a subject to control said first skeletal muscle.

2. The method of claim 1, wherein said first waveform signal is transmitted to said subject's nervous system.

3. A method for controlling skeletal muscles, comprising the steps of:
generating at least a first waveform signal that substantially corresponds to at least one coded waveform signal that is generated in the body and operative in the control of at least a first skeletal muscle; and
transmitting said first waveform signal to a subject to control said first skeletal muscle.

4. The method of claim 3, wherein said first waveform signal is transmitted to said subject's nervous system.

5. A method for controlling skeletal muscles, comprising the steps of:
capturing a plurality of waveform signals generated in the body, said waveform signals being operative in the control of at least a first skeletal muscle;
generating at least a first waveform signal, said first waveform signal including at least a second waveform signal that substantially corresponds to at least one of said captured waveform signals and is operative in the control of said first skeletal muscle; and
transmitting said second waveform signal to a subject to control said first skeletal muscle.

6. The method of claim 5, wherein said second waveform signal is transmitted to said subject's nervous system.

7. A method for controlling skeletal muscles, comprising the steps of:
capturing a plurality of waveform signals generated in the body, said waveform signals being operative in the control of skeletal muscles, said waveform signals including a plurality of signal components;
extracting said signal components of said captured waveform signals;
storing said captured waveform signals and said signal components in a storage medium;
generating a first waveform signal that is operative in the control of at least a first skeletal muscle, said first waveform signal including at least a second waveform signal that substantially corresponds to at least one of said captured waveform signals; and
transmitting said first waveform signal to a subject to control said first skeletal muscle.

8. The method of claim 7, wherein said first waveform signal is transmitted to said subject's nervous system.

9. A method for controlling skeletal muscles, comprising the steps of:
capturing a first plurality of waveform signals generated in a first subject's body, said first plurality of waveform signals including first waveform signals that are operative in the control of skeletal muscles;
generating a base-line skeletal muscle waveform signal from said first waveform signals, said base-line skeletal muscle waveform signal being operative in the control of a first skeletal muscle;
capturing a second plurality of waveform signals generated in said first subject's body, said second plurality of waveform signals including at least a second waveform signal that is operative in the control of said first skeletal muscle;
comparing said base-line skeletal muscle waveform signal to said second waveform signal;
generating a third waveform signal based on said comparison of said base-line skeletal muscle and second waveform signals;
transmitting said third waveform signal to said first subject's body, said third waveform signal being operative in the control of said first skeletal muscle.

10. The method of claim 9, wherein said step of capturing said first plurality of waveform signals comprises capturing said first plurality of waveform signals from a plurality of subjects.

11. The method of claim 9, wherein said third waveform substantially corresponds to said second waveform signal.

12. The method of claim 9, wherein said third waveform substantially corresponds to said base-line skeletal muscle waveform signal.

13. The method of claim 9, wherein said third waveform signal is transmitted to said first subject's nervous system.

14. A method for controlling skeletal muscles, comprising the steps of:
capturing a first plurality of waveform signals generated in a first subject's body, said first plurality of waveform signals including first waveform signals that are operative in the control of skeletal muscles;
storing said first plurality of waveform signals in a first location in a storage medium;
generating a base-line skeletal muscle waveform signal from said first plurality of waveform signals, said base-line skeletal muscle waveform signal being operative in the control of a first skeletal muscle;
capturing a second plurality of waveform signals generated in said first subject's body, said second plurality of waveform signals including at least a second waveform signal that is operative in the control of said first skeletal muscle;
storing said second waveform signal in a second location in said storage medium;
comparing said base-line skeletal muscle waveform signal to said second waveform signal;
generating a third waveform signal based on said comparison of said base-line skeletal muscle and second waveform signal;
transmitting said third waveform signal to said first subject, said third waveform signal being operative in the control of said first skeletal muscle.

15. The method of claim 14, wherein said step of capturing said first plurality of waveform signals comprises capturing said first plurality of waveform signals from a plurality of subjects.

16. The method of claim 14, wherein said third waveform signal is transmitted to said first subject's nervous system.

17. A method for controlling skeletal muscles, comprising the steps of:
monitoring the status of at least a first skeletal muscle of a subject;
providing at least one skeletal muscle status signal in response to a skeletal muscle disorder of said first skeletal muscle;
generating at least a first waveform signal that is operative in the control of said first skeletal muscle in response to said skeletal muscle status signal; and
transmitting said first waveform signal to the subject to mitigate said skeletal muscle disorder.

18. The method of claim 17, wherein said first waveform signal is transmitted to said subject's nervous system.

19. A method for controlling skeletal muscles, comprising the steps of:
capturing waveform signals that are generated in the body and are operative in control of skeletal muscles, said waveform signals including at least a first waveform signal that is operative in the control of a first skeletal muscle;
monitoring the skeletal muscle status of said first skeletal muscle of a subject;
providing at least one skeletal muscle status signal indicative of the status of said first skeletal muscle;
storing said captured waveform signals and skeletal muscle status signal in a storage medium;
generating at least a first waveform that is operative in the control of said first skeletal muscle in response to a skeletal muscle status signal that is indicative of a skeletal muscle disorder; and
transmitting said first waveform signal to said subject to mitigate said skeletal muscle disorder.

20. The method of claim 19, wherein said first waveform signal is generated in response to a component of one of said captured waveform signals that is indicative of a skeletal muscle disorder.

21. The method of claim 19, wherein said first waveform signal is transmitted to said subject's nervous system.

22. A method for controlling skeletal muscles, comprising the steps of:
capturing a first plurality of waveform signals generated in the body that are operative in the control of skeletal muscles;
capturing at least a first waveform signal from a first subject's body that is indicative of a skeletal muscle disorder;
generating a confounding signal that is operative to mitigate said skeletal muscle disorder; and
transmitting said confounding waveform signal to said first subject to mitigate said skeletal muscle disorder.

23. A system for controlling skeletal muscles, comprising:
at least a first signal probe adapted to capture waveform signals from the body, said waveform signals being representative of waveform signals naturally generated in the body and operative in the control of skeletal muscles;
a processor in communication with said signal probe and adapted to receive said waveform signals, said processor being further adapted to generate at least a first waveform signal based on said captured waveform signals, said first waveform signal being recognizable by at least a first skeletal muscle as a control signal; and
at least a second signal probe adapted to be in communication with a subject's body for transmitting said first waveform signal to said subject's body to control said first skeletal muscle.

24. The system of claim 23, wherein said processor includes a storage medium adapted to store said captured waveform signals.

25. The system of claim 23, wherein said second signal probe is adapted to transmit said first waveform signal directly to said subject's body by direct conduction to said subject's nervous system.
